(19) United States
(12) Patent Application Publication
(10) Pub. No.: US 2013/0245716 A1
(43) Pub. Date: Sep. 19, 2013

(54) SLEEVE FOR STIMULATION OF TISSUE REGENERATION

(71) Applicants: Daniel Hechavarria, Boston, MA (US); David Kaplan, Concord, MA (US); Michael Levin, Swampscott, MA (US); Susan Braunhut, Wellesley, MA (US)

(72) Inventors: Daniel Hechavarria, Boston, MA (US); David Kaplan, Concord, MA (US); Michael Levin, Swampscott, MA (US); Susan Braunhut, Wellesley, MA (US)

(73) Assignee: Tufts University, Boston, MA (US)

(21) Appl. No.: 13/643,797

(22) PCT Filed: Apr. 29, 2011
(86) PCT No.: PCT/US11/34435

§ 371 Date: Jun. 5, 2013

Related U.S. Application Data
(60) Provisional application No. 61/329,287, filed on Apr. 29, 2010.

(51) Int. Cl. A61N 1/44

(52) U.S. Cl. CPC A61N 1/44
USPC 607/50

(57) ABSTRACT

The regenerative sleeve encompasses the wound site of an amputated appendage and provides an environment conducive to tissue regeneration. The sleeve includes a tubular reservoir having an outer body that encloses the end of the appendage including the wound site and provides a sealed wound space between the wound site and the outer body. The sleeve also includes a cuff disposed in an opening formed in the outer body, the cuff being configured to fit on the appendage, and an access port disposed on the outer body and configured to allow administration of fluids to the wound space. The sleeve assembly was effective in supporting early stages of murine digit tip regeneration when combined with a porcine urinary bladder matrix (UBM) pepsin digest and electrical stimulation.

### STATEMENT AS TO FEDERALLY SPONSORED RESEARCH

This invention was made with government support under EB002520 awarded by the National Institutes of Health and W911NF-07-1-0572 awarded by the US Army. The government has certain rights in the invention.

### BACKGROUND OF THE INVENTION

Tissue regeneration involves a cascade of biological events that combine to fully rebuild an excision or appendage that was lost during trauma or amputation. There is a distinct difference between a typical wound healing response and a regenerative response. These two processes, while similar in many aspects, result in completely different end products. During the course of normal wound healing, many complex biological structures, such as sweat glands, ducts and hair follicles, cannot be rebuilt since the biological machinery to do so is not available. In a typical adult mammalian skin wound, these structures are not regenerated since development of these tissues and organs require highly specific physiological processes to occur. In addition, normal wound closure and scar formation does not provide an adequate environment for these structures to regenerate. Epimorphic regeneration, on the other hand, is the process during which all original structures are replaced with replications of the originals.

While mammals and most higher vertebrates typically exhibit very limited and time-specific regenerative capacities, there are several model systems that exhibit epimorphic regeneration. These organisms and their ability to perform epimorphic regeneration are heavily studied; however, the exact pathways of regeneration remain obscure. Fortunately, several common motifs among regeneration schemes across a variety of species have been pieced together to generate a solid understanding of the general principles involved in limb regeneration. There are at least three requirements for any system to show epimorphic regeneration. The system must first contain mitotically active cells and, secondly, release signals to promote the proliferation of those cells. Thirdly, the system must be free of factors that can inhibit a regenerative response. These factors may include a dry external environment, bacterial infection, or an overwhelmingly efficient wound healing process that repairs the wound before a regeneration cascade can begin.

As the different stages of epimorphic regeneration progress, not only is there a biochemical response to the trauma, but a biophysical response also occurs that works in tandem with the biochemistry to synergistically repair the defect. The biophysical component of a regenerative response involves both physical mechanics and the various bioelectric events and phenomena such as mass depolarization of cellular membranes and the establishment of minute, long-range electric fields and biological wound currents. Alteration of the cellular transmembrane potential is known to trigger the cell to dedifferentiate and enter a highly mitotic state. Providing a longitudinal electric field is known to both drive an internal wound stump current and provide guidance cues for innervation and the migration of various other cell types near the wound site. While these biophysical phenomena do exist in a normal wound healing response, their presence is significantly more profound in the regeneration process.

### SUMMARY

In some aspects, an apparatus is provided for stimulation of animal tissue regeneration at a wound site disposed on an end of an appendage. The apparatus includes a tubular sleeve, a cuff and an access port. The tubular sleeve includes an outer body that encloses the end of the appendage including the wound site and provides a sealed wound space between the wound site and the outer body. The cuff is disposed in an opening formed in the outer body, and the cuff is configured to conform to the size and shape of the appendage. The access port is disposed on the outer body and is configured to allow transfer of fluids to and from the wound space.

The apparatus may include one or more of the following features: The sleeve further includes an annular seal disposed between the cuff and the outer body, and the seal is configured to support the cuff with respect to the outer body and maintain a sealed closure of the opening. The outer body and cuff are each hollow cylinders, the cuff is disposed at a first end of the outer body, and spacing is provided between the cuff and a second end of the outer body, the second end being opposed to the first end. The access port includes a self-sealing body which closes the second end of the outer body. The outer body and the cuff are substantially coaxial. The apparatus further includes an electrical stimulation device having an anode and a cathode, the anode and cathode are configured to be electrically connected to corresponding terminals of a power source, and a portion of the cathode is disposed in the sleeve. The portion of the cathode is removable from the sleeve.

The apparatus may include one or more of the following additional features: The cuff is resilient. The outer body is resilient. The cuff, outer body and access port are integrally formed whereby the sleeve is a jointless and seamless structure. The access port includes a self-sealing septum which sealingly closes a second opening in the outer body and is configured to maintain a sealed closure of the second opening during and after needle puncture thereof. The access port includes an inlet portion and an outlet portion, the inlet portion configured to be connected to a fluid pump. The outer body is transparent. The outer body is configured to expand in a direction parallel to a longitudinal axis of the appendage. The outer body includes telescoping portions configured to expand the volume of the wound space. The apparatus further includes a rigid outer cover which encloses at least a portion of the sleeve. The apparatus further includes a treatment fluid disposed in the wound space and configured to stimulate tissue regeneration by controlling the ionic properties of cells of the wound site. The treatment fluid is disposed in the wound space and configured to stimulate tissue regeneration by inducing the cells of the wound cite to become mitotically active.

In other aspects, a method of stimulating animal tissue regeneration at a wound site is provided. The method includes the following method steps: Providing an apparatus for stimulation of animal tissue regeneration at a wound site, comprising a sleeve configured to enclose the wound site and provide a sealed wound space between the wound site and the sleeve, the sleeve including an access port configured to allow administration of a fluid to the wound space. Applying the sleeve to the wound site so as to enclose the wound site within the sleeve and form a sealed wound space between the wound site and the sleeve. Treating the wound by including in the wound space a predetermined fluid composition configured to stimulate tissue regeneration.

The method may include one or more of the following features: The predetermined fluid composition is configured to control the ionic properties of cells of the wound site. The predetermined fluid composition is configured to induce wound cells to become mitotically active. The predetermined fluid includes a composition including porcine urinary bladder matrix pepsin digest. The predetermined fluid includes at least two different fluids used sequentially. Moisture is constantly maintained in the wound space. The wound space is filled with the predetermined fluid composition. The predetermined fluid composition is a liquid. The wound site comprises an appendage stump resulting from an amputation of an end of the appendage, the sleeve includes a hollow cylindrical reservoir body, and the access port includes a septum which sealingly closes a first end of the body and is configured to maintain a sealed closure of the first end during and after needle puncture thereof. The sleeve further includes a hollow cylindrical cuff at least partially disposed within the body, and an annular seal disposed between the cuff and the body, and the seal is configured to support the cuff with respect to the body. The cuff is configured to receive the stump therein.

The method may include one or more of the following additional features: The access port includes an inlet portion and an outlet portion, the inlet portion is configured to be connected to a fluid pump, the outlet portion is configured to serve as a drain of the reservoir body, and the step of treating the wound includes providing a continuous flow of the predetermined fluid through the wound space via the inlet and outlet portions. The apparatus further includes an electrical stimulation device including an anode and a cathode configured to be electrically connected to a power source, the cathode being partially disposed within the sleeve. The method further includes applying electrical stimulation to the wound with the electrical stimulation device. The electrical stimulation is conducted to the wound through the predetermined fluid. The electrical stimulation device is configured to mimic electrical signals of biophysiological processes. The application of the electrical stimulation is performed periodically.

The regenerative sleeve provides a closed and controlled macro-environment around a wound or amputation site. By establishing a hydrated and controlled environment, it is possible to stimulate regeneration of tissues, reduce scarring and foster more rapid and direct tissue regeneration.

The regenerative sleeve couples a controlled macro-environment and pharmaceutical treatments with electrical stimulation. The electrical stimulation included with the regenerative sleeve allows for electrical stimulation to occur at the wound site and serves to mimic the biophysical processes of limb regeneration that has been observed in vertebrate systems that spontaneously undergo limb regeneration, such as urodeles, and juvenile frogs. The incorporation of an external power source, a cathode and an anode, provides an electric field aligned with a longitudinal axis of the limb, draws current out of the amputation site at the wound core and replicates the stump currents observed in amphibian models.

An additional form of electrical stimulation is provided by the regenerative sleeve which includes the utilization of a liquid pharmacological treatment composition designed to alter the cellular transmembrane potential of the cells at the wound site to help induce mitotic activity.

An example is provided in which the regenerative sleeve was studied in an animal model. In particular, the regenerative sleeve was used to enclose a surgically amputated murine digit. The regenerative sleeve provided an in utero-type environment conducive to promoting a regenerative response, including a controlled hydrated environment, electrical stimulation at the wound site, and the ability to administer a fluid treatment composition to the wound site to facilitate the recruitment and/or dedifferentiation of cells and blastema formation.

### BRIEF DESCRIPTION OF THE DRAWINGS

FIG. 1 is a perspective view of a regenerative sleeve disposed on a mouse digit.

FIG. 2 is an anatomical diagram of a mouse digit showing an amputation line.

FIG. 3 is an exploded view of the regenerative sleeve of FIG. 1.

FIG. 4 is a side sectional view of the regenerative sleeve of FIG. 1.

FIG. 5 is a diagram of the regenerative sleeve of FIG. 1 including an electrical stimulation device.

FIG. 6 is a side sectional view of the regenerative sleeve of FIG. 1 showing a cathode disposed in the reservoir.

FIG. 7 is a diagram of the regenerative sleeve of FIG. 1 showing an alternative electrical stimulation device configuration.

FIG. 8 is a side sectional view of the regenerative sleeve of FIG. 1 showing an alternative configuration of the cathode disposed in the reservoir.

FIG. 9 is a perspective view of the regenerative sleeve of FIG. 1 showing a syringe and drain disposed in the access port.

FIG. 10 is a diagram of the regenerative sleeve of FIG. 1 used with a fluid pump.

FIG. 11 is a perspective view of an alternative embodiment regenerative sleeve.

FIG. 12 is an exploded view of another alternative embodiment regenerative sleeve.

FIG. 13 is an exploded view of another alternative embodiment regenerative sleeve and a protective shroud.

FIG. 14 is a side sectional view of the regenerative sleeve of FIG. 13 assembled with the shroud showing the connector in an unrolled, extended configuration.

FIG. 15 is a side sectional view of the regenerative sleeve of FIG. 13 assembled with the shroud showing the connector in a retracted, rolled-back configuration.

FIG. 16 is a side view of an another alternative embodiment regenerative sleeve.

FIG. 17 is a diagram of the regenerative sleeve of FIG. 16 including an electrical stimulation device.

FIGS. 18 and 19 illustrate an alternative configuration protective shroud configured for use with the regenerative sleeve of FIG. 16.

FIG. 20 is a histological image of a control wound tissue.

FIGS. 21 and 22 are histological images of regenerative tissues obtained by method of stimulating animal tissue regeneration at a wound site using the regenerative sleeve of FIG. 1.

### DETAILED DESCRIPTION

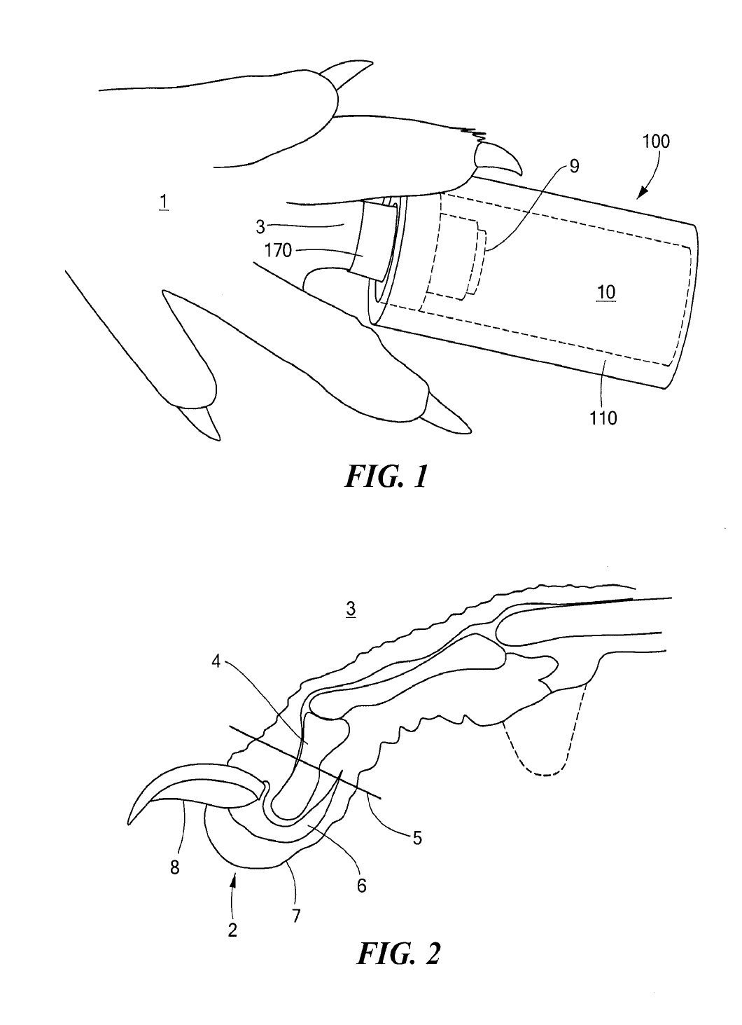

Referring to FIGS. 1 and 2, a regenerative sleeve 100 for stimulating tissue regeneration is shown. The sleeve 100 is used to enclose an end of an appendage, and more particularly to sealingly enclose a wound site 9 corresponding to an amputation of the end of the appendage. In the illustrated embodiment, the sleeve 100 is described with respect to enclosure of mouse digit 3 in which the tip 2 has been amputated along a line 5 through at least a portion of the distal phalange 4, whereby regenerated tissue may include among other tissues, bone tissue, muscle tissue and skin tissue.

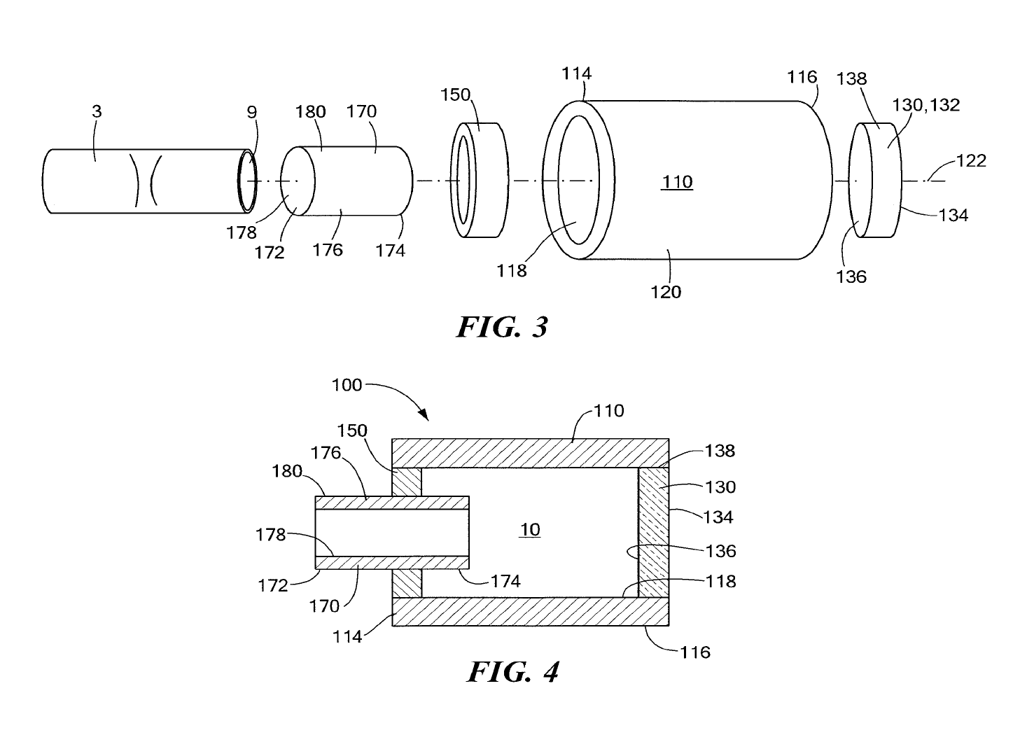

Referring to FIGS. 3 and 4, the sleeve 100 includes a reservoir body 110 and a cuff 170 disposed in an opening in the reservoir body 110. The reservoir body 110 is a hollow cylindrical member that encloses the end of the appendage 3 including the wound site 9 and provides a sealed wound space 10 between the wound site 9 and the reservoir body 110. The reservoir body 110 includes an open first end 114, and an open second end 116 opposed to the first end 114. The reservoir body 110 is transparent to permit observation of the wound site while the sleeve 100 is in use. In addition, the reservoir body 110 is sufficiently rigid to prevent any deflection or indentation of the body walls during use so as ensure that a desired wound space volume is maintained, and to protect the wound site 9. In the illustrated embodiment, the reservoir body 110 is formed of transparent nylon tubing having a length of about 0.25 inches, and an inner diameter of about 0.085 inches.

The reservoir body 110 includes an access port 130 disposed on the reservoir body 110 and configured to allow administration of fluids to, and drainage of fluids from, the wound space 10. The access port 130 is a thin, flat circular disk which is disposed in the open second end 116 of the reservoir body 110, and is sized and shaped to sealingly close the open second end 116. The access port 130 is fitted in the open second end 116 such that a peripheral edge 138 of the access port 130 abuts an inner surface 118 of the reservoir body 110, and an outward facing side 134 of the access port 130 is generally aligned with the end 116 of the reservoir body 110. The access port 130 is formed of a material which is impermeable to gases and liquids, and which is self-sealing. The term self-sealing is used here to describe a material that can be punctured by a needle, which forms a seal about the needle when punctured, and which sealingly closes the puncture hole after the needle is withdrawn. In the illustrated embodiment, the access port 130 is a silicone disk having a thickness of about 0.038 inches.

The sleeve 100 also includes a cuff 170 disposed in the open first end 114 of the reservoir body 110. The cuff 170 is supported within the reservoir body by an annular seal 150. The annular seal 150 is disposed at the first end 114 of the reservoir body 110, and extends between an outer surface 180 of the cuff 170 and the inner surface 118 of the reservoir body 110. The annular seal 150 maintains a sealed closure of the space between cuff 170 and the reservoir body 110 at the first end 114 thereof. The annular seal 150 is resilient, whereby cuffs 170 of varying sizes may be easily accommodated. In some embodiments, the annular seal 150 is formed of silicone.

The cuff 170 is an elongate, thin-walled hollow cylinder which is used to provide a connection between the reservoir body 110 and the appendage 3. The cuff 170 includes a first end 172, a second end 174 that is opposed to the first end 172, and a mid portion 176 disposed between the first and second ends 172, 174. The annular seal 150 supports the cuff 170 with respect to the reservoir body 110 so that the outer surface 180 of the cuff 170 is spaced apart from the inner surface 118 of the reservoir body 110, the mid portion 176 of the cuff 170 resides within a plane defined by the open first end 114 of the reservoir body 110, and the second end 174 of the cuff 170 is spaced apart from the second end 116 of the reservoir body 110. More particularly, the second end 174 of the cuff 170 is spaced apart from an inward facing side 136 of the access port 130. In addition, the cuff 170 is arranged in the reservoir body 110 so that a longitudinal axis of the cuff 170 is coaxial with a longitudinal axis 122 of the reservoir body.

The cuff 170 includes an inner diameter dimensioned to engage and surround a periphery of the appendage 3. The length of the cuff 170 is dependent on the specific application. For example, in use, an adhesive is provided on the inner surface 178 of the cuff 170 to maintain the cuff 170 on the appendage 3, and thus the cuff 170 must be sufficiently long to provide an adhesive bond area sufficient to prevent inadvertent dislocation of the sleeve 100 from the appendage 3. In the illustrated embodiment, the length of the cuff 170 is about 0.10 inches long. In the illustrated embodiment, the cuff 170 is formed of a polyamide tube.

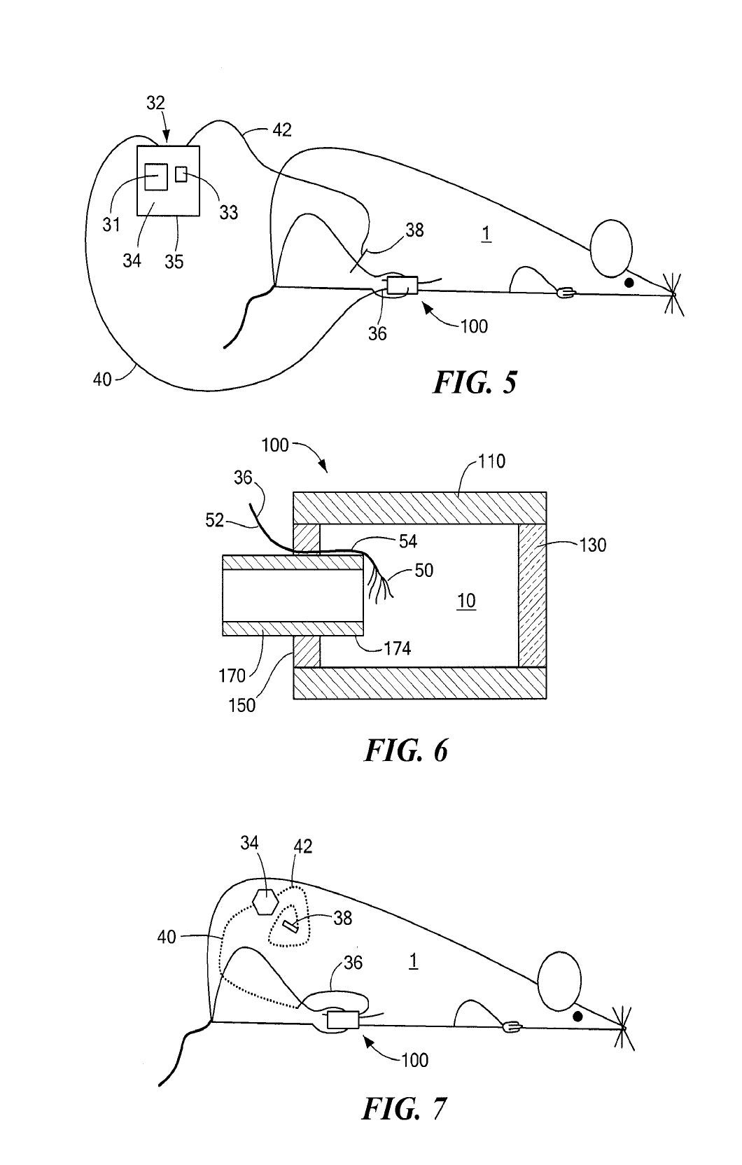

Referring to FIG. 5, in some embodiments, the sleeve 100 also includes an electrical stimulation device 32 to establish a longitudinal electrical field through the wound site 9, which is considered to provide an internal wound stump current and to provide electrical guidance cues for innervation and migration of cell types near the wound site. The electrical stimulation device 32 includes an anode 38 and a cathode 36 that are electrically connected to corresponding terminals of a power source 34 through leads 40, 42.

Referring also to FIG. 6, the cathode 36 is in the form of a stranded stainless steel wire which is disposed in the wound space 10. The cathode 36 includes a distal end 50, a proximal end 52, and a mid portion 54 between the proximal and distal ends 50, 52. In some embodiments, a mid portion of the cathode may be coated, for example with polytetrafluoroethylene such as that sold under the trademark Teflon® manufactured by E.I. du Pont de Nemours and Company, Wilmington, Del., leaving the proximal 52 and distal 50 ends exposed. The proximal end 52 resides outside the sleeve 100 and is connectable to the lead 40. The mid portion 54 extends through the end 114 of the reservoir body 110 between the cuff 170 and the annular seal 150. In some embodiments, the mid portion 54 is fixed at this location using an adhesive (e.g., epoxy). The distal end 50 is disposed within the body 112 of the reservoir body 110 at a location adjacent to the second end 174 of the cuff 170, so that in use, the distal end is closely adjacent to the wound site 9. In some embodiments, wire strands forming the distal end 50 of the cathode 36 are unwound and arranged in a fan shape in order to maximize contact area in the vicinity of the wound site 9.

The anode 38 is a fine conductive wire that may be inserted in the animal 1 at a location distant from the wound site 9. In the illustrated embodiment in which the sleeve 100 is disposed on a mouse digit 3, the anode 38 is disposed in the upper portion of the limb (rear leg) from which the digit 3 extends. In the illustrated embodiment, the anode 38 is a 0.003 inch diameter, uncoated Platinum/Iridium alloy wire which is connected to the power source 32 via the lead 42. The anode can be permanently implanted, or temporarily inserted as needed.

The power source 34 includes a battery pack 31 and circuitry 33, both of which are enclosed in a housing 35 and configured to provide a constant, low level current to the electrodes 36, 38 when connected thereto. In the illustrated embodiment, the power source 34 resides externally of the animal 1, and the electrodes 36, 38 are configured to be detachably connectable to the power source 34. In this arrangement, when electrical stimulation is required, the cathode 36 and anode 38 are electrically connected to the power source 34 for the duration of the electrical stimulation treatment, and then disconnected between electrical stimulation treatments. Since the power source 32 and leads 40, 42 may be detached from the respective electrodes 36, 38, this arrangement conveniently reduces the overall bulk of the combined sleeve 100 and electrical stimulation device 32 during treatment paradigms in which electrical stimulation is used only intermittently.

Referring to FIG. 7, in other embodiments, the cathode 36 and anode 38 may be configured to be permanently connected to the power source 34. This arrangement is suitable for providing a continuous electrical stimulation of the wound site. In such an arrangement, the power source 34 can be configured to be implantable into the animal body, and the electrical leads 40, 42 that connect the cathode 36 and anode 38 to the power source 34 can also be configured to be implanted. By doing so, the overall bulk of the combined sleeve 100 and electrical stimulation device 32 can be reduced.

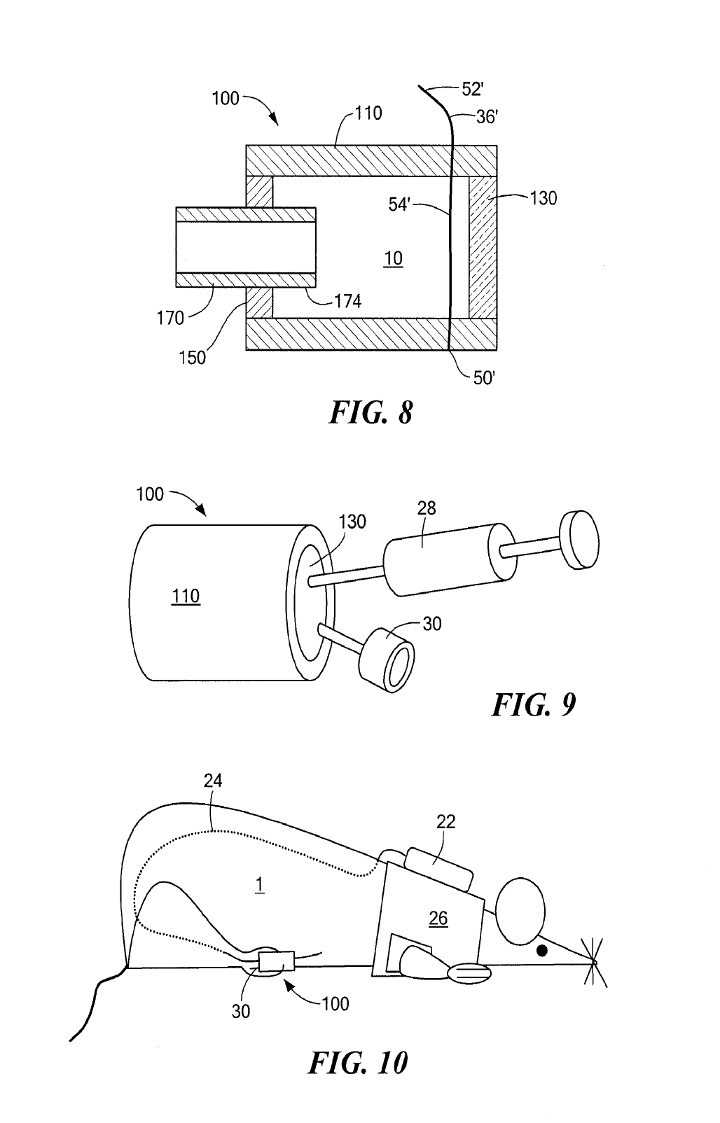

Referring to FIG. 8, an alternative cathode configuration is described. In this embodiment, the cathode 36′ is an uncoated, stranded stainless steel wire that is disposed in the wound space 10 at a location spaced apart from the second end 174 of the cuff 170. In particular, the cathode 36′ is arranged so that the mid portion 54′ extends across the wound space in a direction transverse to the longitudinal axis 122 of the sleeve 100. In this embodiment, the distal end 50′ of the cathode 36′ is fixed to the body 112, and the proximal end 52′ passes through the body 112 at a location that is generally diametrically-opposed to the distal end 50′. By arranging the cathode 36′ at a location spaced apart from the second end 174 of the cuff 170, electrical stimulation applied through the cathode 36′ is conducted indirectly, through the fluids within the wound space 10.

Referring to FIG. 9, the wound space 10 is filled with a fluid to at least maintain a moist wound site 9. More particularly, the wound space 10 is continuously filled with a fluid treatment composition. Applying fluids to the wound space 10 is accomplished by transferring the treatment composition into the wound space 10 through the access port 130 using a syringe 28. A drain 30 may be simultaneously inserted through the access port 130 to permit drainage of wound exudate or treatment fluids from the reservoir body 110, and to permit the wound space to be flushed and/or filled with new treatment fluids.

The fluid treatment composition is formulated to stimulate tissue regeneration by controlling the ionic properties of cells of the wound site and inducing the cells of the wound cite to become mitotically active, as discussed further below. In some embodiments, the treatment composition is applied to wound site immediately upon application of the sleeve 100 to the amputated digit 3. The treatment composition is maintained within the wound space throughout the duration of use of the sleeve 100 due to the sealed configuration of the reservoir body 110. The same treatment fluid may be kept within the reservoir body 110 for the duration of use of the sleeve 100, or the treatment composition may be periodically replaced during the duration of use of the sleeve 100. The replacement fluid may be the original treatment composition, or may consist of a different treatment composition. For example, at least two different treatment compositions may used sequentially. In this case, the selection and ordering of treatment compositions may be determined based at least in part upon the stage of regeneration.

The specific function of the treatment composition is dependent on its particular components. However, the general function of each drug mixture is to chemically stimulate the cells at the wound site 9 to enter a regenerative state. This can be accomplished via several different pathways. For example, the transmembrane potential of the wound cite cells can be targeted to induce the cells to enter a differentiated and highly mitotic state by using depolarization compositions, hyperpolarization combinations, MRL matrix combination, or combinations thereof. Methods and compositions for promoting tissue regeneration by administration of a composition effective to modulate cell membrane potential, for example by increasing intracellular sodium concentration in cellular tissue, are disclosed in U.S. provisional application Nos. 61/273,193, filed Jul. 31, 2009, and 61/227,708, filed Jul. 22, 2009, which are incorporated herein by reference.

An example of a depolarization treatment composition includes sodium (Na⁺) and potassium (K⁺) concentrations of 150 mM and 170 mM, respectively. This is an increase in the normal extracellular ion concentrations for Na⁺ and K⁺ of 150 mM and 5 mM, respectively. The composition is prepared using NaCl and KCl added to basic physiological buffered saline (PBS) solution containing both CA²⁺ and Mg²⁺ to promote normal cell signaling function.

An example of a hyperpolarization treatment composition includes andenosine triphosphate (ATP)-sensitive K+ channel openers such as Pinacidil and Diazoxide. In the presence of ATP, these molecules cause K⁺ channels in the cellular membranes to open up, allowing free K⁺ to flow down a concentration gradient and out of the cell. This outward flux of positive ions causes the transmembrane potential to become more negative, thereby hyperpolarizing the cell.

An example of a Murphy Roths Large (MRL) mouse cell matrix treatment composition is obtained by isolating and decellularizing the extracellular matrix from the blastema-like cells of MRL mice that have received a 2 mm ear punch wound and exposing it to a pepsin digest. MRL mice possess a stunning regenerative capacity compared to other strains of mice, and the MRL regenerative process shows striking resemblances to amphibian processes such as blastema formation.

An example of a urinary bladder matrix treatment composition is obtained by harvesting the extracellular matrix from a porcine urinary bladder and exposing it to a pepsin digest.

Referring to FIG. 10, fluid treatment delivery to the reservoir body 110 is not limited to manual injection. For example, in some embodiments, a continuous flow of a fluid treatment composition into the wound space 10 through the access port 130 is achieved using a fluid pump 22 connected to the access port 130 via tubing 24. In small animal applications where animal mobility and tampering-prevention are of concern, the fluid pump 22 may supported on the animal 1 using a mounting jacket 26. In addition, for the same reasons, the tubing 24 may be at least partially implanted within the animal 1.

Although the illustrated embodiment employs a cuff 170 arranged so that a first end 172 of the cuff is disposed outside the reservoir body 110, the mid portion 176 of the cuff 170 is disposed in the open first end 114 of the reservoir body 110, and the second end 174 is disposed within the interior space of the reservoir body 110, the sleeve 10 is not limited to this arrangement. For example, in some embodiments, the cuff 170 is disposed in the reservoir body 110 so that the first cuff end 172 lies substantially flush with the first end 114 of the reservoir body 110. In other embodiments, the second cuff end 174 lies substantially flush with the first end of the reservoir body.

In the illustrated embodiment, the cuff 170 is formed of a generally inelastic polyamide tube, and the inner diameter of the cuff 170 is dimensioned as required to accommodate appendages 3 of different diameters. In some embodiments, sleeve 10 may be provided in multiple, pre-sized versions. For example, some sleeves 10 may having a cuff 170 of a small inner diameter, while other sleeves 10 may have a cuff 170 of a medium or large inner diameter. In other embodiments, the cuff 170 is formed of an elastic material, whereby one size cuff 170 can accommodate appendages 3 of various sizes.

In the illustrated embodiment, due to the relatively small size of the sleeve 10 used to enclose a wound site on a mouse digit, the electrical stimulation device 30 including the battery pack 32 is housed separately from the sleeve 10, and only the cathode 34 is incorporated into the sleeve assembly. However, for applications in which a sleeve is increased in size to accommodate larger appendages, it is well within the scope of the invention to incorporate the battery pack 32 into the reservoir body 110.

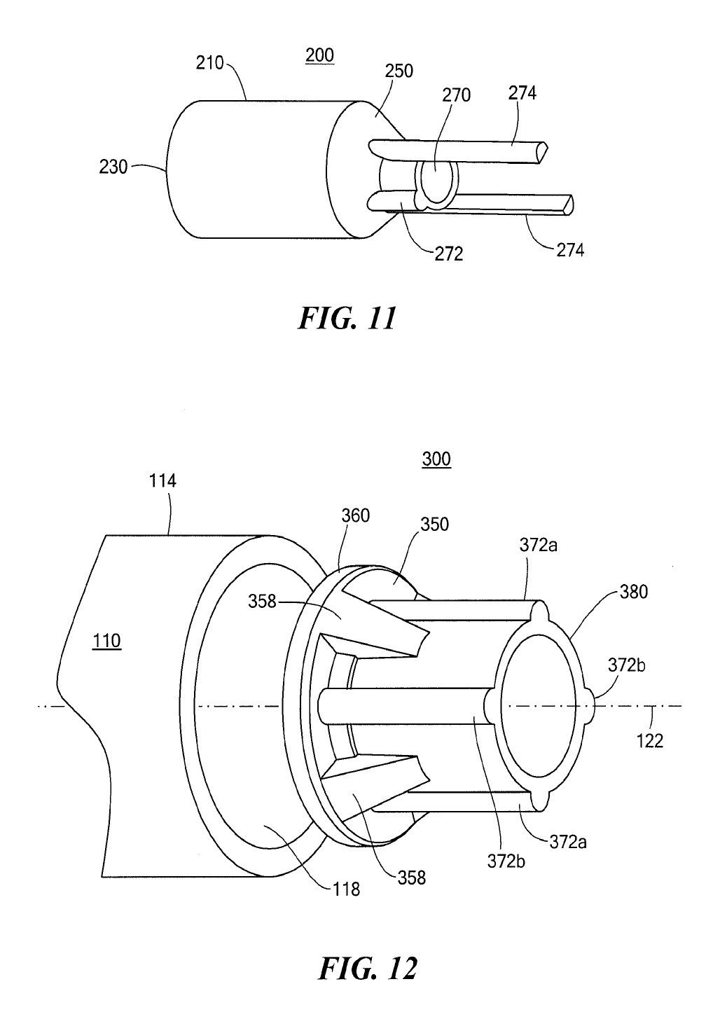

Referring to FIG. 11, an alternative embodiment regenerative sleeve 200 is shown. Similar in form and function to the regenerative sleeve 100 described above, the alterative embodiment regenerative sleeve 200 includes a reservoir body 210, an access port 230, an annular seal 250 and a cuff 270. However, the exterior surface 258 of the annular seal 250 is tapered inward toward the cuff 270, and the outer surface of the cuff 270 includes reinforcing ribs 272. The ribs 272 protrude radially outward and extend along an axial direction of the cuff 270. In addition, the ribs 272 are circumferentially spaced apart. For example, in the illustrated embodiment, ribs 272 are provided on opposed sides of the cuff 270. The outer surface of the cuff 270 is also provided with a pair of opposed, elongated guides 274 which extend along an axial direction of the cuff and protrude axially beyond the cuff end 214. The guides 274 are circumferentially disposed between the ribs 272, and aid in applying the sleeve 200 to the appendage 3. For example, the guides 274 may direct the appendage 3 to the open end of the cuff 270, or may be used to manually grasp and pull the sleeve over the appendage 3.

Unlike the regenerative sleeve 100 described above, in the sleeve 200, the reservoir body 210, access port 230, seal 250 and cuff 270 are all formed integrally of a single piece of material. In addition, the material used to form the sleeve 200 is flexible, permitting the cuff 270 to be applied to appendages 3 of varied sizes, and also permitting elastic expansion of the reservoir body 210. In addition, the material is transparent, self-sealing, easily castable and biocompatible. For example, the material may be a silicon elastomer such as that sold under the registered trademark Dragon Skin®

By forming the regenerative sleeve 200 integrally of a single piece of material, assembly of individual sleeve components is avoided. This is particularly advantageous here due to the very small size of the individual sleeve components. In addition, due to the nature of an amputation wound and the need to apply the treatment fluid to the wound site very quickly after amputation, the difficult and time consuming assembly of the small sleeve components during a surgical procedure is also avoided.

Referring to FIG. 12, an alternative embodiment regenerative sleeve 300 is formed of a reservoir body 110 and access port 130 (not shown in this figure) as described above for the regenerative sleeve 100. In addition, the regenerative sleeve 300 also includes a modified cuff 310 in which a cuff 370 and annular seal 350 are integrally formed of a single piece of material. In the modified cuff 310, the annular seal 350 is disposed at the first end 114 of the reservoir body 110, and extends between an outer surface 380 of the cuff 370 and the inner surface 118 of the reservoir body 110. The annular seal 350 maintains a sealed closure of the space between the modified cuff 310 and the first end 114 of the reservoir body 110. The annular seal 350 includes circumferentially-spaced buttresses 358 that extend between a periphery 360 of the annular seal 350 and an outer surface of the cuff 370. The buttresses 358 protrude radially outward from the outer surface of the cuff 370, extend along an axial direction of the cuff 370, and taper inward from the periphery 360 of the annular seal to the outer surface of the cuff 370. In addition, the outer surface of the cuff 370 includes reinforcing ribs 372. The ribs 372 protrude radially outward and extend along an axial direction of the cuff 370. In addition, the ribs 372 are circumferentially spaced apart. For example, in the illustrated embodiment, two pairs of ribs 372a, 372b are provided, the ribs 372 of a given pair being disposed on opposed sides of the cuff 370. The buttresses 358 and ribs 372 provide axial and radial structural reinforcement to the modified cuff 310.

Like the preceding embodiment, the modified cuff 310 is formed of a flexible material, permitting the cuff 270 to be applied to appendages 3 of varied sizes. In addition, the modified cuff 310 is formed of a material that is easily castable and biocompatible. For example, the material may formed of Dragon Skin® silicon elastomer.

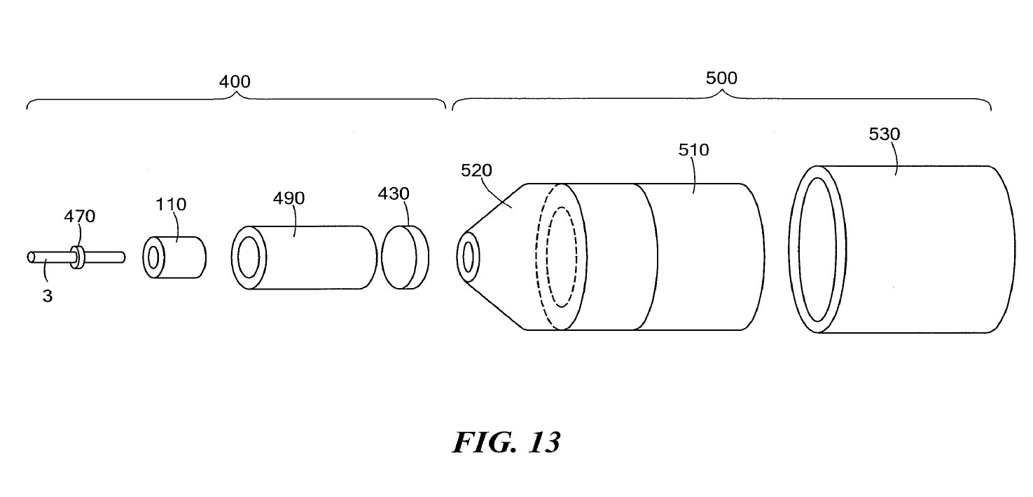

Referring to FIG. 13, an alternative embodiment regenerative sleeve 400 is formed of the reservoir body 110, a supplemental reservoir 490, an access port 430, and a cuff member 470. The supplemental reservoir 490 is a transparent, rigid cylindrical body that includes an open first end 494, and an open second end 496 opposed to the first end 494. The supplemental reservoir 490 surrounds at least the second end 116 of the reservoir body 110, and is dimensioned to be press fit on the outer surface 120 of the reservoir body 110 so that the reservoir body 110 and supplemental reservoir 490 are coaxially arranged. In addition, the supplemental reservoir 490 is axially longer than the reservoir body 110. In this embodiment, the second end 116 of the reservoir body 110 remains open, and the access port 430 is disposed in the second end 496 of the supplemental reservoir 490. The access port 430 is substantially the same in function and structure as the access port 130 described above, but has been increased in size to provide a sealed closure to the second end 496 of the supplemental reservoir 490.

In the regenerative sleeve 400, the cuff member 470 is substantially modified relative to earlier embodiments. In particular, the cuff member 470 is formed of an annular-shaped medical grade foam material. The outer diameter of the cuff member 470 is dimensioned to correspond to the inner diameter of the reservoir body 110. Due to the resilient compliance of the cuff member material, the inner diameter of the cuff member 470 can be made less than the outer diameter of the appendage 3 to ensure a good seal and fit about the appendage 3 while still providing wearer comfort and avoiding pressure-related tissue damage. Moreover, the soft foam accommodates any increases or decreases in swelling of the appendage while maintaining a fluid-sealed closure of the first end 114 of the reservoir body 110.

By providing the reservoir body 110 with the superstructure 490, the volume of the wound space is increased, permitting larger volumes of the treatment composition to be applied to the wound site 9. In addition, due to the press-fit, coaxial arrangement of the reservoirs 110, 490, the overall length of the regenerative sleeve 400 can be increased by telescopically sliding the supplemental reservoir 490 axially relative to the reservoir 100. In particular, this adjustment can be made while the sleeve 400 is in use, for example to accommodate regenerative tissue growth and/or to further adjust the volume of the wound space.

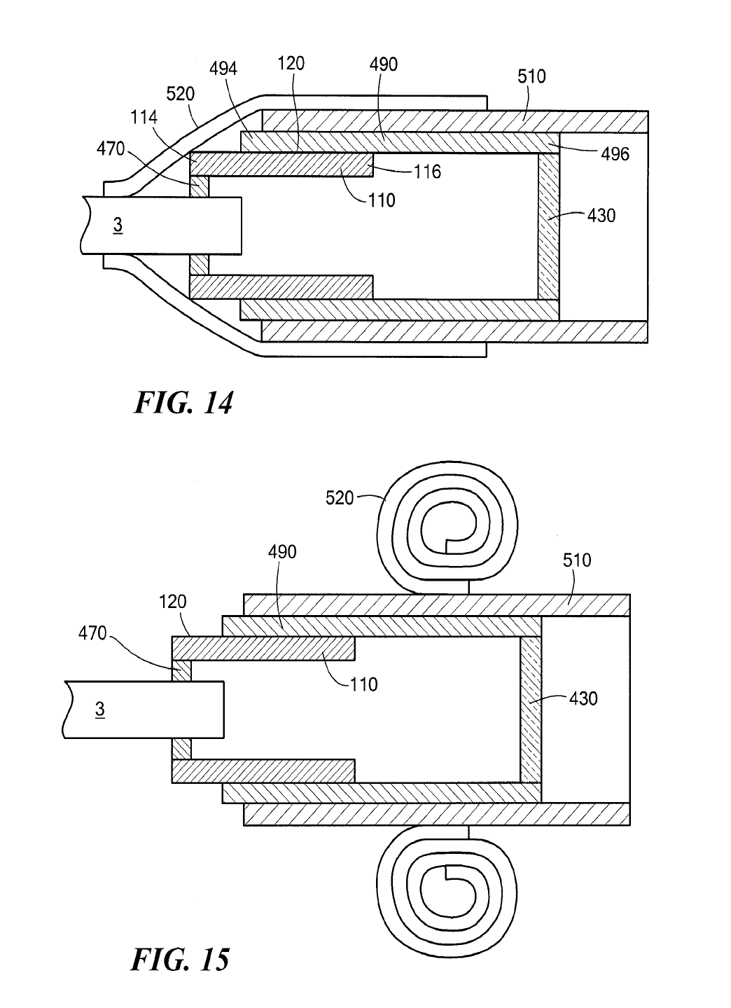

With reference to FIGS. 14 and 15, in some applications in which the regenerative sleeve 400 is used in small mammals, it can be advantageous to provide a transparent protective shroud 500 over the regenerative sleeve 400 to protect it from animal tampering. The shroud 500 includes a transparent cylindrical jacket 510 dimensioned to surround the superstructure 490 of the regenerative sleeve 400, and further includes a flexible connector 520 used to secure the jacket 510 to the appendage 3. The connector 520 is formed of a flexible tubing which is disposed on a first end 514 of the jacket 510, and extends longitudinally from the jacket first end 514 toward appendage 3. For example, the connector 520 may be formed of a silicone tube.

The connector 520 can conveniently be rolled back on itself (FIG. 15) to simplify assembly, and then can be unrolled (FIG. 14) to cover at least a portion of the appendage 3. Although illustrated here as connecting the jacket 510 to the mouse digit 3, this arrangement is not limiting. For example, by providing the connector 520 with sufficient length, the connector 520 can be used provide a connection to the animal limb proximal to the digit 3, for example by surrounding the limb at, or proximal to, the paw.

Referring again to FIG. 13, in some embodiments the shroud 500 further includes a rigid cylindrical shell 530 which can surround the connector 520, leaving the transparent jacket 510 uncovered to permit viewing of the wound site. The shell 500 is used to protect the soft connector 520 from animal tampering.

In the illustrated embodiment, the regenerative sleeve 100, 200, 300, 400 has been described with respect to enclosure of an amputated digit 3 of a mouse 1. However, the sleeve is not limited to this application and may be adapted, for example through appropriate scaling of components, for use on appendages other than digits, as discussed further below. Moreover, the sleeve may be adapted for use on appendages of larger animals, and for use on non-appendage wound sites. In addition, the sleeve may be useful in managing growth at wound sites that do not originate from a traumatic injury such as limb amputation. For example, the sleeve may be useful for treatment of organs or appendages that are insufficient due to birth defect, disease or infection.

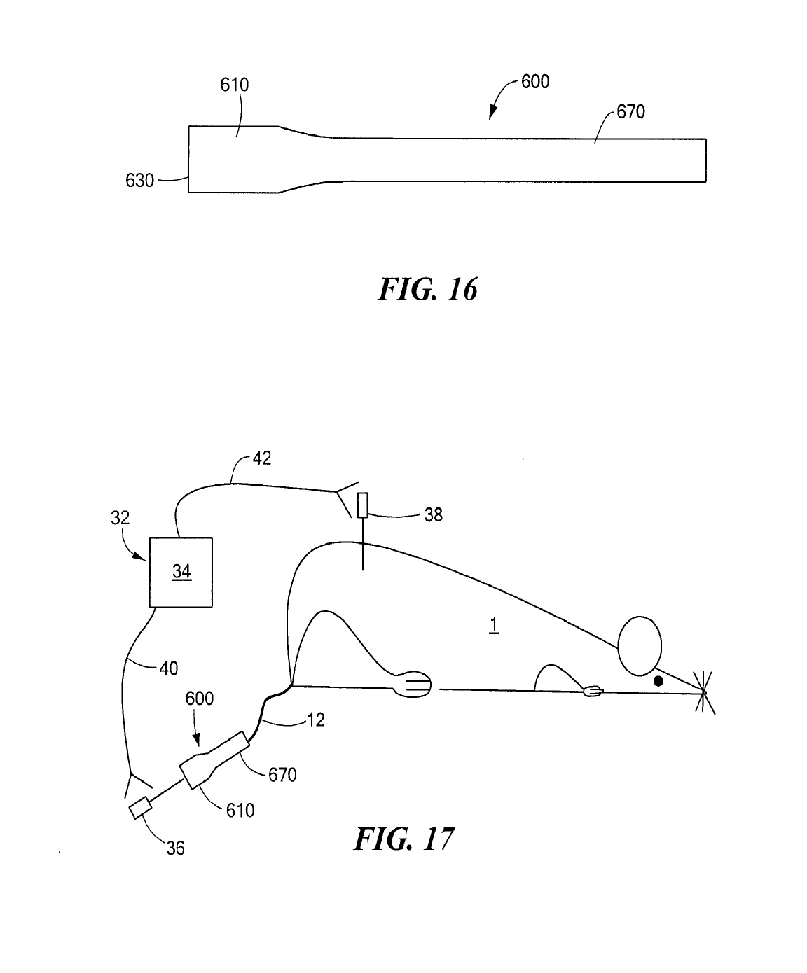

Referring to FIG. 16, another embodiment regenerative sleeve 600 is shown which is particularly adapted for use on a murine tail 12. The regenerative sleeve 600, like the regenerative sleeve 200 is formed integrally of a single cast piece of material, and includes an elongated cuff 670 sized and shaped to receive a length of the tail 12, and a reservoir portion 610 connected to the cuff 670. The reservoir 610 is a hollow cylindrical member that encloses the amputated end of the tail 12 including the wound site 9 and provides a sealed wound space 10 between the wound site 9 and the reservoir 610. The reservoir 610 may be transparent to permit observation of the wound site while the sleeve 600 is in use.

Referring to FIG. 17, the regenerative sleeve 600 may include an electrical stimulation device 32 to establish a longitudinal electrical field through the wound site 9. As described above in more detail, the electrical stimulation device 32 includes an anode 38 and a cathode 36 that are electrically connected to corresponding terminals of a power source 34 through corresponding leads 40, 42.

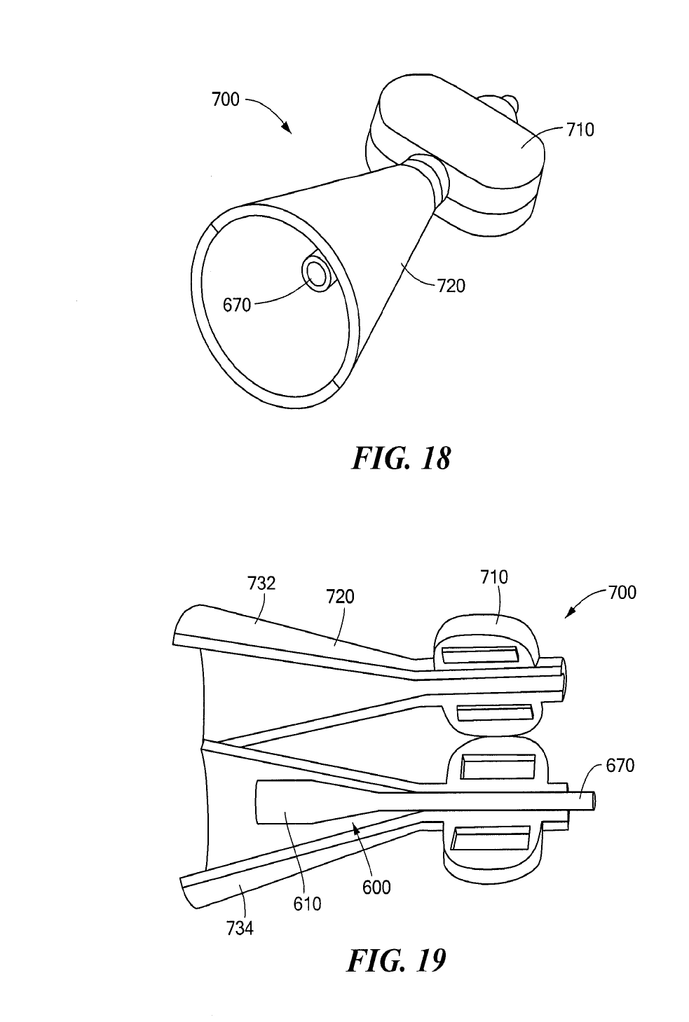

Referring to FIGS. 18 and 19, a protective shroud 700 may be provided that is adapted to prevent animal tampering with the regenerative sleeve 600. The protective shroud 700 is formed of a tough, lightweight material such as plastic, and includes a base portion 710 which encloses the cuff 670 of the sleeve 600, and further includes a conical shaped collar 720 which protrudes from an end of the base portion 710, and which surrounds the reservoir 610 of the sleeve 600. The collar 720 extends from the base portion 710 to a location that is distal to the reservoir 610, making difficult for the animal to gain access to the sleeve 600. The conical collar 720 is arranged so that the widest portion is located at the distal end of the sleeve 600. This arrangement advantageously permits easy access to the access port 630 so that treatment fluids and electrical stimulation can be administered to the wound site 9. The shroud 700 is provided as two halves 732, 734 hinged along one axial side, permitting the shroud 700 to be opened as shown in FIG. 19 to receive the regenerative sleeve 600 while use on the murine tail 12. Once the sleeve 600 is received in the shroud 600, the halves 732, 734 are assembled as shown in FIG. 18 and fixed, for example by using adhesive.

A method of stimulating animal tissue regeneration at a wound site 9 using the regenerative sleeve 100, 200, 300, 400, 600 will now be described. In the method described herein, the renerative sleeve is used to enclose a wound site 9 formed by amputation of the end 2 of a murine digit 3. In particular, the amputation is provided along an amputation line 5 through the distal phalange 4 of a murine digit 3 as shown in FIG. 2.

The method of stimulating animal tissue regeneration at a wound site 9 includes the following method steps:

The method includes providing a regenerative sleeve 100, 200, 300, 400, 600 as described above. The regenerative sleeve is applied to the end of the digit 3 immediately following amputation of the end 2 of the digit 3, so as to enclose the wound site 9 within the sleeve and form a sealed wound space between the wound site 9 and the sleeve.

The method includes treating the wound site 9 by filling the wound space 10 with a predetermined fluid composition configured to maintain a moist wound site 9, and to stimulate tissue regeneration at the wound site 9. In some embodiments, the fluid composition is administered by puncturing the access port 130 with a pair of hypodermic syringes, where one syringe is used to deliver the fluid composition to the wound space 10 and the other syringe is open-ended and serves as a vent (FIG. 9). During this step, care is taken to prevent formation of air bubbles within the wound space 10.

The predetermined fluid composition is configured to control the ionic properties of cells of the wound site to induce wound cells to become mitotically active. In some embodiments, the predetermined fluid composition includes porcine urinary bladder matrix pepsin digest, but this is not limiting. Treating the wound may include continuous treatment of the wound site 9 with a single fluid composition throughout the duration of use of the sleeve. Alternatively, the treatment composition may be periodically replaced during the duration of use of the sleeve. The replacement fluid may be the original treatment composition, or may consist of a different treatment composition. For example, at least two different treatment compositions may used sequentially. In this case, the selection and ordering of treatment compositions may be determined based at least in part upon the stage of regeneration. In some embodiments, a continuous flow of the predetermined fluid through the wound space may be provided using fluid pump.

The method further includes treating the wound site 9 by applying electrical stimulation to the wound site 9 using the electrical stimulation device 32. Electrical stimulation can be achieved by directly contacting the wound site with a cathode 36. Alternatively, electrical stimulation can be achieved disposing the cathode 36 in the wound space 10 whereby the electrical stimulation is conducted to the wound through the treatment fluid. The electrical stimulation is applied in such a way as to mimic electrical signals of biophysiological processes, in terms of current intensity (up to 10 uA maximum), flow direction, and temporal pattern. In some embodiments, application of the electrical stimulation is performed periodically. For example, stimulation can be provided on alternating days for a duration of 15-30 minutes on each of those days. In other embodiments, application of the electrical stimulation is performed continuously.

Although in the method described herein, the regenerative sleeve is used to enclose a wound site 9 formed by amputation of the end of a murine digit 3, this is not limiting. For example, it is well within the scope of the invention to apply the disclosed method to amputation wounds of other appendages, to such wounds in the appendages of other animals, and to wounds not resulting from amputation (for example, crush injuries).

### EXAMPLE

### Animal Study

The effectiveness of the regenerative sleeve 10 described above to biochemically and biophysically stimulate tissue regeneration in a murine toe amputations was studied.

In particular, twelve 6-8 week old male mice, weighing approximately 20-25 g. were used in this study. Even though female mice are known to demonstrate slightly more developed regenerative capacities, males were selected since the majority of the intended long-term beneficiaries of this study are injured male soldiers than have suffered limb loss in conflict.

The mice were anesthetized via intraperitoneal injection with Ketamine (90-120 mg/kg) and Xylazine (10 mg/kg) prior to surgery. Lubricating eye drops are administered to the eyes of sedated mice to prevent dehydration. After anesthetization, the mice were prepared for surgery by repeatedly cleaning the right hind foot with 70% ethanol and then with a 10% povidone iodine solution. Fur near the anode insertion site is removed with an electric trimmer and razor. The surgical sites are also cleaned with ethanol and povidone iodine after fur removal. The digit amputation was performed at the midline of the 2nd phalange of the right, hind middle digit (digit 3, Standard US Nomenclature) with extra-fine bone scissors. Amputations took place under a Leica EZ 4D microscope. Scissors and other surgical instruments were sterilized between mice using alcohol and a hot glass bead sterilizer. Digit tips were properly discarded after amputation. The surgical time for each mouse averaged between 10 and 15 minutes from the onset of sedation, excluding time required for electrical stimulation.

This investigation involved two treatment groups containing six mice each (see Table 1). Group 1 received a Regenerative sleeve 10 containing the urinary bladder matrix (UBM) digest control treatment. Group 2 received a Regenerative sleeve 10 containing the UBM digest treatment. Electrical stimulation was provided to all subjects on days 0, 1 and 3.

TABLE 1
Treatment Matrix

| Group | Liquid Treatment | n | Electrical Stimulation |
|---|---|---|---|
| 1 | UBM digest control | 6 | 6.4 uA for 15 minutes on |
| | (neutralized pepsin | | days 0, 1 and 3 |
| | buffer) | | |
| 2 | UBM digest | 6 | 6.4 uA for 15 minutes on |
| | | | days 0, 1 and 3 |

The UBM pepsin digest treatment and control treatment, a neutralized pepsin buffer, were the only treatments administered. The UBM treatments have been shown to perform well as scaffolds and promotion of regenerative healing. The preparation of the UBM treatments was accomplished by harvesting the ECM from a porcine urinary bladder and, then exposing the ECM to a pepsin mediated enzymatic deigestion. While a fully characterized composition the UBM digest remains unknown, various molecules such as collagen, glycosoaminoglycans (GAGs), matrix metalloproteinases (MMPs) and a variety of growth factors are present and serve as a physical scaffold for cell growth.

### Treatment Delivery:

The ECM digest treatments were administered by puncturing the distal silicone septum of the Regenerative sleeve 10 with a pair of 30.5 gauge hypodermic syringes. One syringe was used to deliver the liquid cocktail treatment while the second syringe was open-ended and serves as a vent, allowing the air within the Regenerative sleeve 10 reservoir to escape as treatment is added. At this time, the system is checked for leaks. Care was taken to avoid the formation of air bubbles within the Regenerative sleeve 10 reservoir. Electrical stimulation was administered while the animals were sedated. Immediately prior to electrical stimulation, the 0.18 mm diameter electrical stimulation acupuncture needle was inserted into the haunch ipsilateral with the Regenerative sleeve 10. This anode was inserted at an angle to ensure it did not pierce the muscle layer. The stainless steel cathode was built in to the Regenerative sleeve 10 reservoir as shown in FIG. 1. By definition, electrical current flows from the anode towards the cathode.

The electrical connection was made to the anode using a mini alligator clip, and the anode was promptly removed and discarded after stimulation. Electrical stimulation began promptly after the anode was inserted. After the power supply was adjusted to deliver the proper current (6.4 uA), the mouse was electrically connected to the power supply and stimulated for 15 minutes. To check for proper function of the electrical stimulation system, the initial current flow was verified at the start of the electrical stimulation session using an in-line Ammeter. After electrical stimulation, the mouse was relocated from the surgical area to the heated stimulation area for recovery. Per protocol, mice received buprenorphine as an analgesic to reduce pain following the surgical procedures. The buprenorphine was administered subcutaneously at 0.05 mg/kg immediately after all surgical procedures have taken place.

### Recovery and Euthanasia:

Recovery from surgery and electrical stimulation occurred on a heating pad. Mice were intermittently monitored by visual analysis and toe-pinch reflex to determine level of consciousness. After the mice began to move on their own, they were placed in individual housing containers for the duration of the study with free access to food and water in a temperature controlled room. During the initial bedding period, soft Kimwipe® bedding was provided. Euthanasia was performed by CO2 inhalation prior to collections of samples for histology.

### Histology:

On day fourteen, the amputated digits along with an adjacent digit for a control were isolated for histology. Due to the presence of the bone in the tissue samples, all samples are decalcified using the Decalcifier I® treatment (Surgipath Inc.). After decalcification, the samples were placed in 10% formalin until they were paraffin embedded. The digits were then sectioned along the proximal-distal axis, stained with trichrome, and imaged at low and high magnifications.

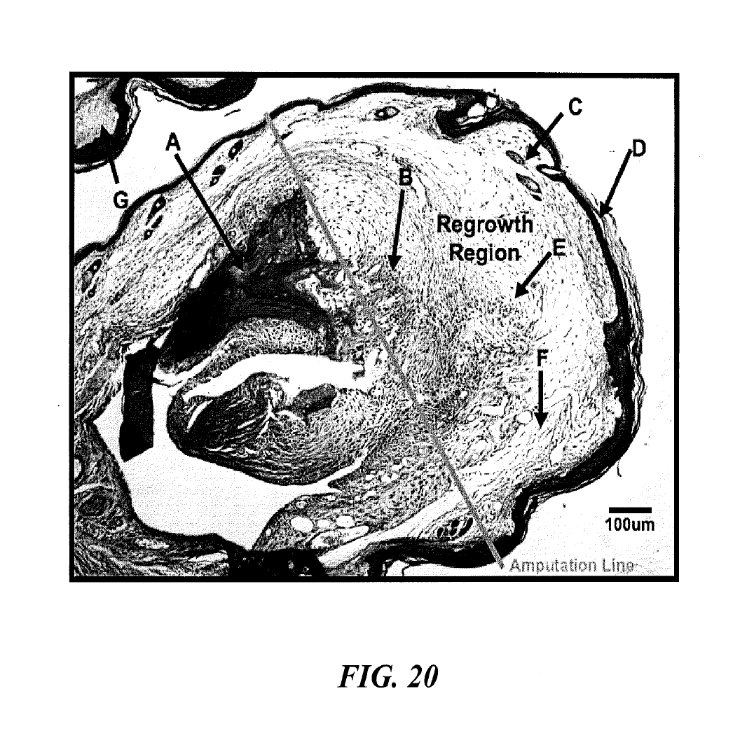

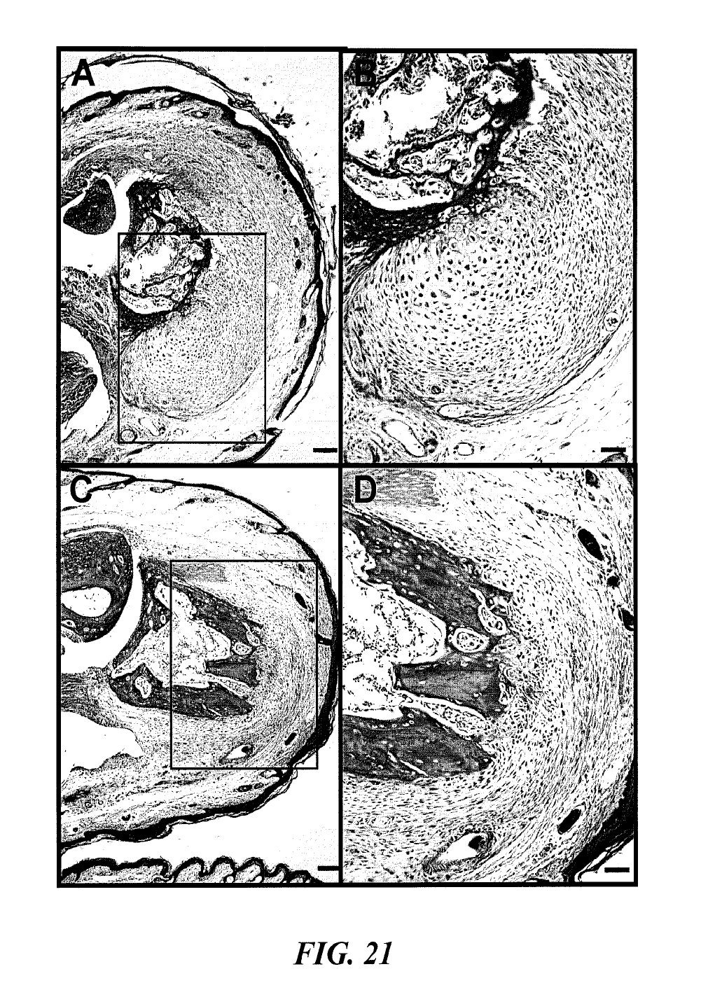

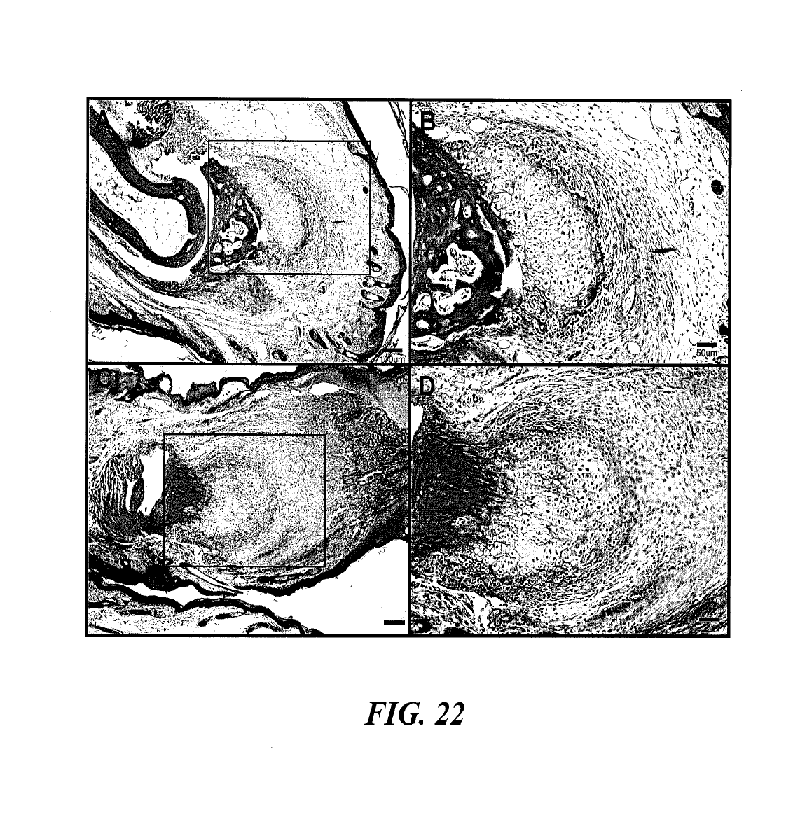

TABLE 2
Result Summary

| Treatment | Day 14 Result Summary | Images |
|---|---|---|
| True Control | Lymphocytes: significant presence | FIG. 20 |
| (−) Regenerative sleeve 10 | of cells Wound epithelium | |
| (−) electrical stimulation | and new glands: relatively thin | |
| (−) pharmacological | wound epithelium with minimal | |
| treatment | new gland formation within | |
| | the re-growth region. | |
| | Large mononuclear eosinophilic | |
| | cells: significant presence of | |
| | LMECs without any indication | |
| | of advanced organization | |
| UBM pepsin digest control | Lymphocytes: low presence of | FIG. 21 |
| (+) Regenerative sleeve 10 | immune cells Wound epithelium | |
| (+) UBM pepsin digest | and new glands: slightly thicker | |
| control cocktail | wound epithelium with increased | |
| (+) electrical stimulation | new gland formation within | |
| | the re-growth regions | |
| | Large mononuclear eosinophilic | |
| | cells: strong presence of LMECs | |
| | with some samples showing | |
| | increased organization and | |
| | formation of possible lacuna-type | |
| | regions | |
| UBM pepsin digest | Lymphocytes: low presence of | FIG. 22 |
| (+) Regenerative sleeve 10 | immune cells Wound epithelium | |
| (+) UBM pepsin digest | and new glands: thickest wound | |
| cocktail | epithelium with strongest | |
| (+) electrical stimulation | evidence of new gland formation | |
| | and vascularization within | |
| | the re-growth region. | |
| | Large mononuclear eosinophilic | |
| | cells: Strongest presence of | |
| | LMEC's with a high degree of | |
| | organization and lacuna-type | |
| | regions adjacent to original bone. | |

The regenerative sleeve 100 was designed to address the following issues in a murine model system: wound site hydration, drug delivery, electrical stimulation, subject ambulation and stress management, tamper prevention, and simplicity of installation. The materials and configuration for the device were chosen to minimize potential damage caused by gnawing, scratching, normal movement and exposure to the rodent housing environment. The regenerative sleeve 100 was streamlined to minimize complexity of the device for fabrication and handling reasons and to reduce the number and severity of possible complications that could arise during any part of the surgical procedure.

Study results indicate that the regenerative sleeve 100 provides a protected and hydrated environment by encompassing the wound site. Qualitative analysis of the histological data indicates that the presence of the regenerative sleeve's 100 well-hydrated environment plays a crucial role in enhancing digit regeneration. Also, administration of electrical stimulation to the wound site enhances this response to the extent where highly organized structures indicative of bone remodeling were observed as early as day 14 in most subjects. Subject receiving a regenerative sleeve 100 with UBM pepsin digest control solution with electrical stimulation showed evidence of enhanced regeneration (FIG. 21) over the control digit (FIG. 20). FIG. 20 shows a histological image take 14 post amputation in a Subjects receiving a regenerative sleeve 100 with UBM pepsin digest and electrical stimulation exhibited an even greater evidence of regeneration over the UBM control treatment as indicated by a more pronounced network of collagen deposition and large eosinophilic mononuclear cells (FIG. 22). FIG. 20 shows a histological image taken 14 post amputation on a C57bl/6 mouse. This mouse was a true control and received no treatment (pharmacological or physical) or wound dressing post amputation. The distal region of the digit is indicated near point (D), and the adjacent digit is indicated at (G). The amputation line shows the approximate line of amputation on Day 0, and the regrowth region is defined as the region of tissue distal to the amputation line. (A) Original, mature bone; (B) Proliferative large mononuclear eosinophilic cells; (C) New hair follicle and sebaceous glands; (D) Wound epithelium; (E) Lympyhocytes; (F) New collagen disposition, possibly scar tissue formation. FIGS. 21 and 22 show (A, C) Low and (B, D) high magnification images of two different digit tips in the UBM pepsin digest control with electrical stimulation group. Scale bars: 100 um (A,C), 50 um (B, D).

A selected illustrative embodiment of the invention is described above in some detail. It should be understood that only structures considered necessary for clarifying the present invention have been described herein. Other conventional structures, and those of ancillary and auxiliary components of the system, are assumed to be known and understood by those skilled in the art. Moreover, while a working example of the present invention has been described above, the present invention is not limited to the working example described above, but various design alterations may be carried out without departing from the present invention as set forth in the claims.

What is claimed is:

1. An apparatus for stimulation of animal tissue regeneration at a wound site disposed on an end of an appendage, comprising:
a tubular sleeve including
an outer body that encloses the end of the appendage including the wound site and provides a sealed wound space between the wound site and the outer body;
a cuff disposed in an opening formed in the outer body, the cuff configured to conform to the size and shape of the appendage; and
an access port disposed on the outer body and configured to allow transfer of fluids to and from the wound space.

2. The apparatus of claim 1 wherein the sleeve further includes an annular seal disposed between the cuff and the outer body, the seal configured to support the cuff with respect to the outer body and maintain a sealed closure of the opening.

3. The apparatus of claim 1 in which
the outer body and cuff are each hollow cylinders,
the cuff is disposed at a first end of the outer body, and
spacing is provided between the cuff and a second end of the outer body, the second end being opposed to the first end.

4. The apparatus of claim 3 wherein the access port includes a self-sealing body which closes the second end of the outer body.

5. The apparatus of claim 3 wherein the outer body and the cuff are substantially coaxial.

6. The apparatus of claim 1 further comprising an electrical stimulation device including an anode and a cathode,
the anode and cathode configured to be electrically connected to corresponding terminals of a power source, and
a portion of the cathode being disposed in the sleeve.

7. The apparatus of claim 6 wherein the portion of the cathode is removable from the sleeve.

8. The apparatus of claim 1 wherein the cuff is resilient.

9. The apparatus of claim 1 wherein the outer body is resilient.

10. The apparatus of claim 1 wherein the cuff, outer body and access port are integrally formed whereby the sleeve is a jointless and seamless structure.

11. The apparatus of claim 1 wherein the access port includes a self-sealing septum which sealingly closes a second opening in the outer body and is configured to maintain a sealed closure of the second opening during and after needle puncture thereof.

12. The apparatus of claim 1 wherein the access port comprises an inlet portion and an outlet portion, the inlet portion configured to be connected to a fluid pump.

13. The apparatus of claim 1 wherein the outer body is transparent.

14. The apparatus of claim 1 wherein the outer body is configured to expand in a direction parallel to a longitudinal axis of the appendage.

15. The apparatus of claim 1 wherein the outer body includes telescoping portions configured to expand the volume of the wound space.

16. The apparatus of claim 1 further comprising a rigid outer cover which encloses at least a portion of the sleeve.

17. The apparatus of claim 1 further comprising a treatment fluid disposed in the wound space and configured to stimulate tissue regeneration by controlling the ionic properties of cells of the wound site.

18. The apparatus of claim 1 further comprising a treatment fluid disposed in the wound space and configured to stimulate tissue regeneration by inducing the cells of the wound cite to become mitotically active.

19. A method of stimulating animal tissue regeneration at a wound site, the method comprising the following method steps:
providing an apparatus for stimulation of animal tissue regeneration at a wound site, comprising a sleeve configured to enclose the wound site and provide a sealed wound space between the wound site and the sleeve, the sleeve including an access port configured to allow administration of a fluid to the wound space;
applying the sleeve to the wound site so as to enclose the wound site within the sleeve and form a sealed wound space between the wound site and the sleeve;
treating the wound by including in the wound space a predetermined fluid composition configured to stimulate tissue regeneration.

20. The method of claim 19 wherein the predetermined fluid composition is configured to control the ionic properties of cells of the wound site.

21. The method of claim 19 wherein the predetermined fluid composition is configured to induce wound cells to become mitotically active.

22. The method of claim 19 wherein the predetermined fluid comprises a composition including porcine urinary bladder matrix pepsin digest.

23. The method of claim 19 wherein the predetermined fluid comprises at least two different fluids used sequentially.

24. The method of claim 19 wherein moisture is constantly maintained in the wound space.

25. The method of claim 19 wherein the wound space is filled with the predetermined fluid composition.

26. The method of claim 19 wherein the predetermined fluid composition is a liquid.

27. The method of claim 19, wherein
the wound site comprises an appendage stump resulting from an amputation of an end of the appendage, and
the sleeve includes
a hollow cylindrical reservoir body;
the access port including a septum which sealingly closes a first end of the body and is configured to maintain a sealed closure of the first end during and after needle puncture thereof;
a hollow cylindrical cuff at least partially disposed within the body, and
an annular seal disposed between the cuff and the body, the seal configured to support the cuff with respect to the body,
wherein the cuff is configured to receive the stump therein.

28. The method of claim 19 wherein
the access port comprises an inlet portion and an outlet portion, the inlet portion configured to be connected to a fluid pump, the outlet portion configured to serve as a drain of the reservoir body, and
the step of treating the wound includes providing a continuous flow of the predetermined fluid through the wound space via the inlet and outlet portions.

29. The method of claim 19 wherein the apparatus further includes an electrical stimulation device including an anode and a cathode configured to be electrically connected to a power source, the cathode being partially disposed within the sleeve.

30. The method of claim 29, wherein the method further includes applying electrical stimulation to the wound with the electrical stimulation device.

31. The method of claim 30 wherein the electrical stimulation is conducted to the wound through the predetermined fluid.

32. The method of claim 30 wherein the electrical stimulation device is configured to mimic electrical signals of biophysiological processes.

33. The method of claim 30 wherein the application of the electrical stimulation is performed periodically.
